(19) United States
(12) Patent Application Publication
(10) Pub. No.: US 2024/0075274 A1
(43) Pub. Date: Mar. 7, 2024

(54) Cap for Closed Male Luer Connector

(71) Applicant: Becton, Dickinson and Company, Franklin Lakes, NJ (US)

(72) Inventor: Karthik Bhaskar, Coimbatore (IN)

(21) Appl. No.: 18/242,858

(22) Filed: Sep. 6, 2023

Related U.S. Application Data
(60) Provisional application No. 63/404,370, filed on Sep. 7, 2022.

(51) Int. Cl. A61M 39/20

(52) U.S. Cl. CPC A61M 39/20

(57) ABSTRACT

A cap for a closed male luer connector having a housing and a male luer, with the closed male luer connector having a closed position where a fluid path through the closed male luer connector is closed and an open position where a fluid path through the closed male luer connector is open, the cap including a body having a first end and a second end positioned opposite the first end, with the body defining an opening configured to receive a portion of the male luer of the closed male luer connector. The body is configured to actuate the closed male luer connector from the closed position to the open position when the cap is fully inserted into the closed male luer connector. The body is configured to expand in a radial direction.

### CROSS-REFERENCE TO RELATED APPLICATION

The present invention claims priority to U.S. Provisional Application Ser. No. 63/404,370 entitled “Cap for Closed Male Luer Connector” filed Sep. 7, 2022, the entire disclosure of which is hereby incorporated by reference in its entirety.

### BACKGROUND OF THE INVENTION

### Field of the Disclosure

The present disclosure relates generally to a cap for a closed male luer connector.

### Description of the Related Art

Health care providers reconstituting, transporting, and administering hazardous drugs, such as cancer treatments, can put health care providers at risk of exposure to these medications and present a hazard in the health care environment. Unintentional chemotherapy exposure can affect the nervous system, impair the reproductive system, and bring an increased risk of developing blood cancers in the future. Some drugs must be dissolved or diluted before they are administered, which involves transferring a solvent from one container to a sealed vial containing the drug in powder or liquid form, by means of a needle. Drugs may be inadvertently released into the atmosphere in gas form or by way of aerosolization, during the withdrawal of the needle from the vial and while the needle is inside the vial if any pressure differential between the interior of the vial and the surrounding atmosphere exists. In order to reduce the risk of health care providers being exposed to toxic drugs, the transfer of these drugs is accomplished utilizing a closed system transfer device or system. Closed system transfer devices or systems may include syringe adapters, patient connectors, IV bag spikes, and vial adapters.

### SUMMARY OF THE INVENTION

In one aspect or embodiment, a cap for a closed male luer connector having a housing and a male luer, with the closed male luer connector having a closed position where a fluid path through the closed male luer connector is closed and an open position where a fluid path through the closed male luer connector is open, includes a body having a first end and a second end positioned opposite the first end, with the body defining an opening configured to receive a portion of the male luer of the closed male luer connector. The body is configured to actuate the closed male luer connector from the closed position to the open position when the cap is fully inserted into the closed male luer connector. The body is configured to expand in a radial direction.

The second end of the body may be configured to engage a valve member of the closed male luer connector to actuate the closed male luer connector from the closed position to the open position when the cap is fully inserted into the closed male luer connector. The cap may include an engagement member configured to engage the male luer and a portion of the housing of the closed male luer connector when the cap is partially and fully inserted into the closed male luer connector. The engagement member may be a tab extending from the second end of the body. The engagement member may include first and second tabs extending from the body, with the first tab spaced from the second tab. The body may define a slit extending in a direction parallel to a longitudinal axis of the cap. The body may define a first slit and a second slit positioned opposite the first slit, with the first and second slits each extending in a direction parallel to a longitudinal axis of the cap. The body may define a recessed portion on an inner surface of the body defining the opening. The body may include a plurality of external ribs extending in a direction parallel to a longitudinal axis of the cap.

In a further aspect or embodiment, a closed male luer connector includes a housing, a male luer, a female luer, with the closed male luer connector having a closed position where a fluid path through the closed male luer connector is closed and an open position where a fluid path through the closed male luer connector is open, and a cap according to any of the aspects or embodiments discussed above. The cap has a first position where the cap is engaged with the male luer, a portion of the male luer is received within the opening of the cap, and the closed male luer connector is in the closed position, and a second position where the closed male luer connection is in the open position. The second position is spaced from the first position in a direction extending parallel to a longitudinal axis of the cap.

The closed male luer connector may include a valve member having an extension positioned radially outward from the male luer, with the second end of the cap engaged with the extension when the cap is in the first and second positions. A portion of the valve member may be configured to extend through a seal when the closed male luer connector is moved from the closed position to the open position. Movement of the cap from the first position to the second position may cause the second end of the cap to move the valve member and to extend a portion of the valve member through the seal. The body of the cap may engage the male luer and radially expand as the cap is moved from the first position to the second position.

### BRIEF DESCRIPTION OF THE DRAWINGS

The above-mentioned and other features and advantages of this disclosure, and the manner of attaining them, will become more apparent and the disclosure itself will be better understood by reference to the following descriptions of aspects of the disclosure taken in conjunction with the accompanying drawings, wherein:

FIG. 1 is a schematic view of a conventional infusion set and closed male luer connector.

FIG. 2 is a top view of the closed male luer connector of FIG. 1.

FIG. 3 is a side view of the closed male luer connector of FIG. 1.

FIG. 4 is a cross-sectional view of the closed male luer connector of FIG. 1, showing a closed position of the closed male luer connector.

FIG. 5 is a cross-sectional view of the closed male luer connector of FIG. 4, showing an open position of the closed male luer connector.

FIG. 6 is a perspective view of a cap for a closed male luer connector according to one aspect or embodiment of the present application.

FIG. 7 is a cross-sectional view of the cap of FIG. 6, showing the cap in a first position with respect to a closed male luer connector.

FIG. 8 is a perspective view of the cap of FIG. 6, showing the cap in a second position with respect to a closed male luer connector.

FIG. 9 is a side view of the cap of FIG. 6, showing the cap in a second position with respect to a closed male luer connector.

FIG. 10 is a cross-sectional view of the cap of FIG. 6, showing the cap in a second position with respect to a closed male luer connector.

Corresponding reference characters indicate corresponding parts throughout the several views. The exemplifications set out herein illustrate exemplary aspects of the disclosure, and such exemplifications are not to be construed as limiting the scope of the disclosure in any manner.

### DETAILED DESCRIPTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

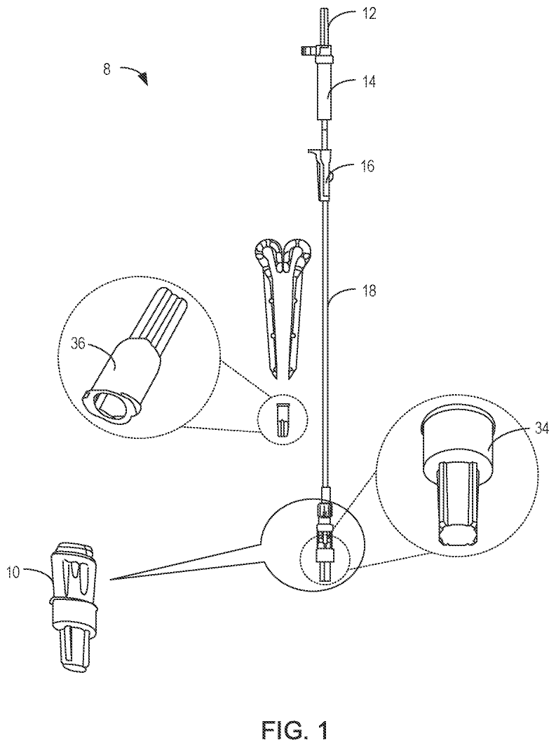

Referring to FIG. 1, in order to prevent hazardous drugs from leaking or dripping, infusion line sets 8 can include a closed male luer connector 10, which remains closed until engaged with a corresponding female luer connector. Such infusion sets 8 may include an intravenous bag spike adapter 12, drip chamber 14, line clamp 16, and a length of tubing 18.

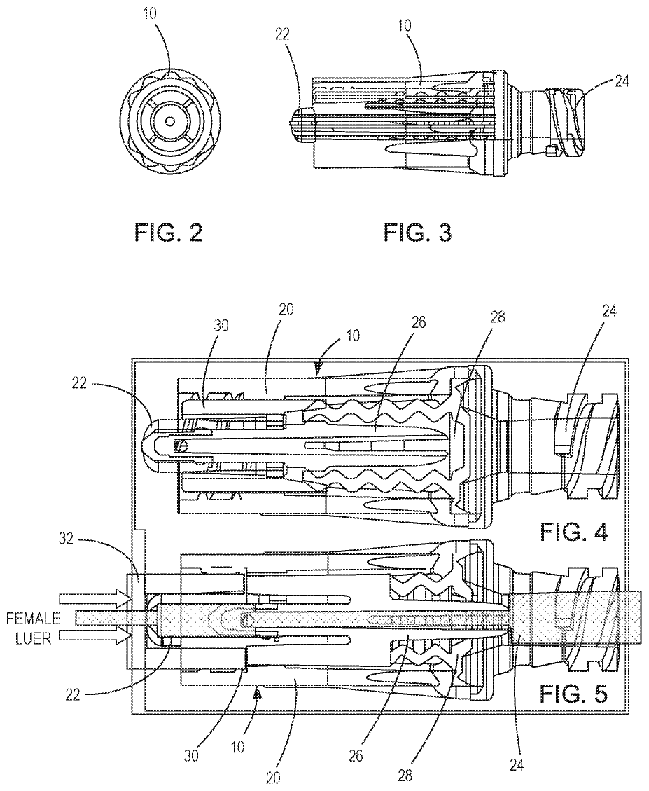

Referring to FIGS. 2-5, the closed male luer connector 10 includes a housing 20, a male luer 22, a female luer 24, and a valve member 26. The valve member 26 is movable or actuatable from a closed position (FIG. 4) to an open position (FIG. 5). A portion of the valve member 26 extends through a seal 28 when the valve member 26 is moved from the closed position to the open position, which opens the fluid path extending through the closed male luer connector 10. A portion of the valve member 26 is received within the male luer 22, with movement of the valve member 26 between the closed position to the open position opening and closing a passageway extending through the closed male luer connector 10. The valve member 26 includes extensions 30, which engage a corresponding female luer 32, as shown in FIG. 5, to move the valve member 26 to the open position when the corresponding female luer 32 is connected to the closed male luer connector 10. The extensions 30 and the valve member 26 may be formed integrally. The closed male luer 10 may be the Texium™ closed male luer commercially available from Becton, Dickinson and Company.

Referring to FIGS. 1 and 5, the closed male luer connector 10 may include a cap 34 to maintain sterility and to avoid contamination of the fluid path. However, in order to prime the infusion set, a separate priming cap 36 needs to be used, which actuates the closed male luer connector 10 to move the male luer 22 from the closed position to the open position to open the fluid path thereby allowing the infusion set 8 and closed male luer connector 10 to be primed.

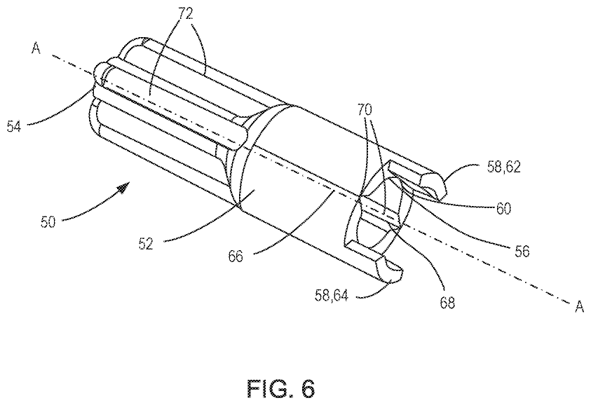

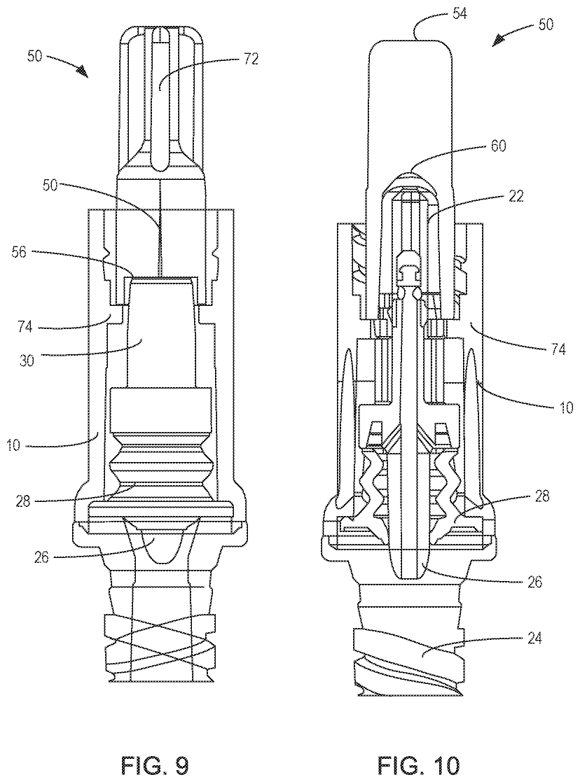

Referring to FIGS. 6-10, according to one aspect or embodiment of the present application, a cap 50 for use with the closed male luer connector 10 includes a body 52 having a first end 54 and a second end 56 positioned opposite the first end 54. As discussed in more detail below, the cap 50 is configured to maintain the sterility of the closed male luer connector 10 prior to use of the closed male luer connector 10 and also enable the cap 50 to be utilized to prime the closed male luer connector 10 and any associated components of an infusion set.

Referring again to FIGS. 6-10, the body 52 of the cap 50 defines an opening 60 configured to receive a portion of the male luer 22 of the closed male luer connector 10. The body 52 is configured to actuate the closed male luer connector 10 from the closed position to the open position when the cap 50 is fully inserted into the closed male luer connector 10. The body 52 is configured to expand in a radial direction, which is described in additional detail below. In one aspect or embodiment, the cap 50 includes an engagement member 58 configured to engage the male luer 22 and a portion of the housing 20 of the closed male luer connector 10. In one aspect or embodiment, the engagement member 58 includes first and second tabs 62,64 extending from the body 52, with the first tab 62 spaced from the second tab 64. The first and second tabs 62,64 extend from the second end 56 of the body 52, although other suitable arrangements may be utilized. Further, although two tabs are shown, the engagement member 58 may include one or more tabs 62,64. In one aspect or embodiment, the engagement member 58 is configured to increase a surface area of contact with the male luer 22 to ensure the cap 50 is securely retained on the closed male luer connector 10 during shipping and storage and also is configured to limit axial movement of the cap 50. The second end 56 of the body 52 of the cap 50 is configured to engage of the extensions 30 of the closed male luer connector 10 when the cap 50 is inserted into the closed male luer connector 10.

The body 52 of the cap 50 is configured to expand radially outward due to the body 52 defining a first slit 66 and a second slit 68 positioned opposite the first slit 66, with the first and second slits 66,68 each extending in a direction parallel to a longitudinal axis A of the cap 50. In other words, a portion of the cap 50 can expand radially outward in a direction perpendicular to the longitudinal axis A of the cap 50 due to the slits 66,68 allowing movement of a portion of the body 52. The first and second slits 66,68 each extend from the second end 56 of the cap 50 to a position intermediate the first and second ends 54,56, although other suitable arrangements may be utilized. Although two slits are shown, the body 52 may include one or more slits 66,68. In further aspects or embodiments, rather than providing the slits 66,68, the cap 50 includes a thin film of plastic that permanently tears when the cap 50 is fully inserted into the closed male luer connector 10. The thin film may include an initiation point, such as a crack or weakened portion, at the second end 56 to reduce the initial resistance to expand radially. As shown in FIG. 6, the body 52 defines recessed portions 70 on an inner surface of the body 52 defining the opening 60. The recessed portions 70 are V-shaped, although other suitable arrangements may be utilized. The recessed portions 70 are positioned adjacent to the slits 66,68. The body 52 also includes a plurality of external ribs 72 extending in a direction parallel to the longitudinal axis A of the cap 50. The plurality of ribs 72 provide a gripping surface for a person to grasp and manipulate the cap 50.

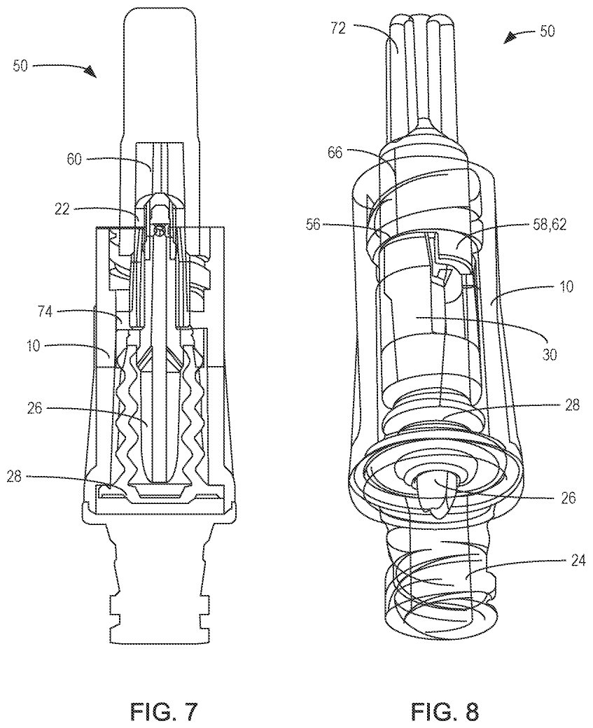

Referring to FIGS. 7-10, the cap 50 has a first position (FIG. 7) where the cap 50 is engaged with the male luer 22, a portion of the male luer 22 is received within the opening 60 of the cap 50, and the closed male luer connector 10 is in the closed position, and a second position (FIGS. 8-10) where the closed male luer connector 10 is in the open position. The second position is spaced from the first position in a direction extending parallel to the longitudinal axis A of the cap 50. The second end 56 of the cap 50 is engaged with the extensions 30 of the male luer 22 when the cap 50 is in the first and second positions. Movement of the cap 50 from the first position to the second position causes the second end 56 of the cap 50 to move the extensions 30 and the valve member 26, with a portion of the valve member 26 extending through the seal 28. The body 52 of the cap 50 radially expands due to increasing diameter of the male luer 22 as the cap 50 is moved from the first position to the second position.

Accordingly, prior to packaging of the closed male luer connector 10, the closed male luer connector 10 and the cap 50 may be sterilized, with the cap 50 partially inserted into the housing 20 of the closed male luer connector 10 until the cap 50 is in the first position. When the cap 50 is in the first position, the cap 50 and the closed male luer connector 10 are packaged. When the closed male luer connector 10 is ready to be used, the closed male luer connector 10 and the cap 50 are removed from the packaging and connected to the infusion set 8. The cap 50 remains in place in the first position to maintain the sterility of the male luer 22 of the closed male luer connector 10 and the other components of the infusion set 8. Prior to priming the infusion set 8 and the closed male luer connector 10, the cap 50 is moved to the second positon by pushing or moving the cap 50 further into the housing 20 of the closed male luer connector 10 in a direction extending parallel to the longitudinal axis A of the cap 50. The cap 50 may be pushed into the closed male luer connector 10 until the engagement member 58 or other portion of the cap 50 engages a flange 74 extending inward from the housing 20 and/or until the male luer 22 reaches an end of the opening 60. When the cap 50 is moved to the second position, the cap 50 engages the extensions 30 of the valve member 26 of the closed male luer connector 10 to move the closed male luer connector 10 to the open position thereby allowing the closed male luer connector 10 and infusion set 8 to be primed. After priming, the cap 50 can be removed by pulling the cap 50 opposite to the direction utilized to move the cap 50 to the second position, and the male luer 22 of the closed male luer connector 10 can be connected to a patient catheter or a primary infusion set.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims. To the extent possible, one or more features of any aspect or embodiment discussed above can be combined with one or more features of any other aspect or embodiment.

What is claimed is:

1. A cap for a closed male luer connector having a housing and a male luer, the closed male luer connector having a closed position where a fluid path through the closed male luer connector is closed and an open position where a fluid path through the closed male luer connector is open, the cap comprising:
a body having a first end and a second end positioned opposite the first end, the body defining an opening configured to receive a portion of the male luer of the closed male luer connector, the body configured to actuate the closed male luer connector from the closed position to the open position when the cap is fully inserted into the closed male luer connector, wherein the body is configured to expand in a radial direction.

2. The cap of claim 1, wherein the second end of the body is configured to engage a valve member of the closed male luer connector to actuate the closed male luer connector from the closed position to the open position when the cap is fully inserted into the closed male luer connector.

3. The cap of claim 1, further comprising an engagement member configured to engage the male luer and a portion of the housing of the closed male luer connector when the cap is partially and fully inserted into the closed male luer connector.

4. The cap of claim 3, wherein engagement member comprises a tab extending from the second end of the body.

5. The cap of claim 3, wherein the engagement member comprises first and second tabs extending from the body, the first tab spaced from the second tab.

6. The cap of claim 1, wherein the body defines a slit extending in a direction parallel to a longitudinal axis of the cap.

7. The cap of claim 1, wherein the body defines a first slit and a second slit positioned opposite the first slit, the first and second slits each extending in a direction parallel to a longitudinal axis of the cap.

8. The cap of claim 6, wherein the body defines a recessed portion on an inner surface of the body defining the opening.

9. The cap of claim 1, wherein the body comprises a plurality of external ribs extending in a direction parallel to a longitudinal axis of the cap.

10. A closed male luer connector comprising:
a housing;
a closed male luer;
a female luer, the closed male luer connector having a closed position where a fluid path through the closed male luer connector is closed and an open position where a fluid path through the closed male luer connector is open; and
a cap having a body having a first end and a second end positioned opposite the first end, the body defining an opening configured to receive a portion of the closed male luer, the body configured to actuate the closed male luer connector from the closed position to the open position when the cap is fully inserted into the closed male luer connector, wherein the body is configured to expand in a radial direction;
the cap having a first position where the cap is engaged with the male luer, a portion of the male luer is received within the opening of the cap, and the closed male luer connector is in the closed position, and a second position where the closed male luer connection is in the open position, wherein the second position is spaced from the first position in a direction extending parallel to a longitudinal axis of the cap.

11. The closed male luer connector of claim 10, further comprising a valve member having an extension positioned radially outward from the male luer, the second end of the cap engaged with the extension when the cap is in the first and second positions.

12. The closed male luer connector of claim 11, wherein a portion of the valve member is configured to extend through a seal when the closed male luer connector is moved from the closed position to the open position.

13. The closed male luer connector of claim 12, wherein movement of the cap from the first position to the second position causes the second end of the cap to move the valve member and to extend a portion of the valve member through the seal.

14. The closed male luer connector of claim 10, wherein the body of the cap engages the male luer and radially expands as the cap is moved from the first position to the second position.
